(19) United States
(12) Patent Application Publication
(10) Pub. No.: US 2011/0118659 A1
(43) Pub. Date: May 19, 2011

(54) MULTI-PORT SYRINGE ASSEMBLY

(76) Inventors: Armand Maaskamp, Coto de Caza, CA (US); Asim Syed, Irvine, CA (US); Ryan Maaskamp, San Francisco, CA (US)

(21) Appl. No.: 12/617,992

(22) Filed: Nov. 13, 2009

(51) Int. Cl. A61M 5/31; A61M 1/00

(52) U.S. Cl. 604/28; 604/30

(57) ABSTRACT

A multi-port syringe assembly that allows fluid to be drawn into or injected out of the assembly. For example, the assembly may include one syringe that is used to aspirate fluid from a patient and another syringe to inject a medical fluid into the patient. As one medical application, the first syringe can withdraw fluid from a patient and the second syringe can be used to inject a steroid into the patient. The assembly may include a third syringe so that a second medical fluid can be injected into the patient. With a three syringe assembly an anesthetic can be injected into the patient before aspiration of fluid. Alternatively, the assembly may have a two-way valve located in the first proximal channel.

### BACKGROUND OF THE INVENTION

1. Field of the Invention

The present application relates to a valve connector for a multi-port syringe assembly.

2. Prior Art

Patients with rheumatoid arthritis are sometimes treated with an injection of a steroid directly into the tendon sheath of the patient's hand. When correctly administered the steroid can reduce inflammation and improve mobility of the hand. It has been found that steroids can cause atrophy of tissue surrounding the tendon sheath. When extracting the syringe needle some of the steroid may leak into the surrounding tissue and cause patient discomfort. One technique employed to reduce steroid leakage is to unscrew the syringe from the needle while the needle is still in the hand, attach a syringe full of anesthetic, and inject some anesthetic into the tendon. This is done to clear steroid out the needle before the syringe needle is pulled out of the hand. Having to unscrew one syringe and attaching another syringe to the inserted needle is a cumbersome technique for injecting anesthetic.

### BRIEF SUMMARY OF THE INVENTION

A connector valve assembly for a plurality of syringes. The connector includes a housing that has a pair of proximal ports, and a distal port in fluid communication with the proximal ports through first and second proximal channels. The assembly may have a first one-way valve located within the first proximal channel to allow fluid flow in a first direction and a second one-way valve located within the second proximal channel that allows fluid flow in an opposite second direction. Alternatively, the assembly may have a two-way valve located in the first proximal channel.

### BRIEF DESCRIPTION OF THE DRAWINGS

FIG. 1 is an illustration of a syringe assembly;

FIG. 2 is an enlarged partially exploded view of the syringe assembly;

FIG. 3 is an illustration of an alternate embodiment of the syringe assembly; and,

FIG. 4 is an illustration of another alternate embodiment of the syringe assembly that has three syringes;

FIG. 5 is an illustration of another alternate embodiment of a syringe assembly with a manually actuated on/off valve.

### DETAILED DESCRIPTION

Disclosed is a multi-port syringe assembly that allows fluid to be drawn into or injected out of the assembly. For example, the assembly may include one syringe that is used to aspirate fluid from a patient and another syringe to inject a medical fluid into the patient. As one medical application, the first syringe can withdraw fluid from a patient and the second syringe can be used to inject a steroid into the patient. The assembly may include a third syringe so that a second medical fluid can be injected into the patient. With a three syringe assembly an anesthetic can be injected into the patient before aspiration of fluid. Alternatively, the assembly may have a two-way valve located in the first proximal channel. The two-way valve allows for the ejection of fluid from the first syringe when in a first position and the aspiration of fluid into the first syringe when in a second position.

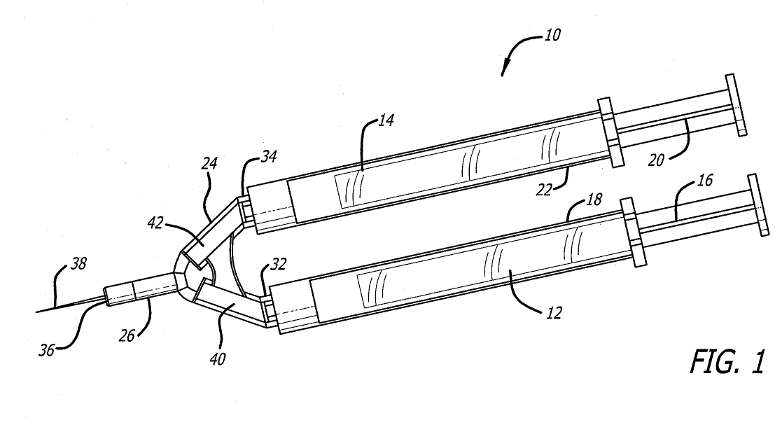

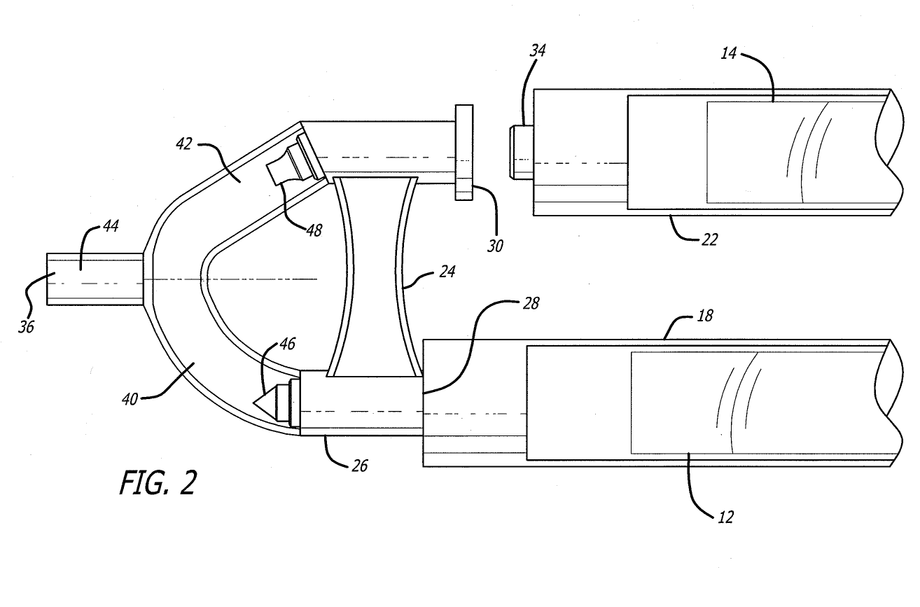

Referring to the drawings more particularly by numbers, FIGS. 1 and 2 show an embodiment of a dual syringe assembly 10. The assembly 10 includes a first syringe 12 and a second syringe 14. The first syringe 12 may have a plunger 16 that can move inside a tube 18. Likewise, the second syringe 14 may have a plunger 20 that is moved inside a tube 22. The tubes 18 and 22 can be constructed from transparent or semi-transparent plastic material and have markings (not shown) that provide an indication of the volume of fluid within the syringe. The plungers 16 and 20 can also be constructed from a plastic material.

The assembly 10 may include a connector valve assembly 24 attached to the syringes 12 and 14. The connector 24 includes a housing 26 that has a first proximal port 28 and a second proximal port 30. The first proximal port 28 is connected to a flange 32 of the first syringe 12. The second proximal port 30 is connected to a flange 34 of the second syringe 14. The housing 26 and flanges 32 and 34 are constructed so that a user can easily connected and disconnect the syringes 12 and 14 from the connector assembly 24.

The connector assembly housing 26 has a distal port 36 that is in fluid communication with a needle 38 that is attached to the housing 26. The distal port 36 and needle 38 are in fluid communication with the syringes 12 and 14 through a pair of proximal channels 40 and 42 and a distal channel 44. Each channel 40 and 42 may have a check valve 46 and 48, respectively. The check valves 46 and 48 prevent fluid from one syringe 12 or 14 from being injected into the other syringe 14 or 12. By way of example, the check valves 46 and 48 may be duck-bill valves constructed from a rubber or plastic material. The connector 24 may be constructed from a molded plastic material.

The syringes 12 and 14 can be arranged in a parallel relative relationship as shown, or can be attached to the connector 24 so as to be at an oblique angle relative to each other. One of the syringes 12 or 14 can contain a steroid and the other syringe 14 or 12 may contain an anesthetic. The assembly 10 may be used to sequentially inject the steroid and anesthetic into a hand.

In operation, a user can attach the needle 38 and syringes 12 and 14 to the connector assembly 24. The needle 38 can be attached to the connector assembly 24 before or after attachment of the syringes 12 and 14 to the assembly 24. The needle 38 can then be inserted into a patient. The plunger 16 of the first syringe 12 is depressed to inject a first medical fluid into the patient through the needle 38. The first medical fluid flows through the first one-way valve 46 but is blocked from flowing into the second syringe 14 by the second one-way valve 48. By way of example, the first medical fluid may be a steroid. The plunger 20 of the second syringe 14 is then depressed to inject a second medical fluid into the patient. The second medical fluid flows through the second one-way valve 48 but is blocked from flowing into the first syringe 14 by the first one-way valve 46. By way of example, the second medical fluid may be an anesthetic. The needle 38 is then pulled out of the patient's hand.

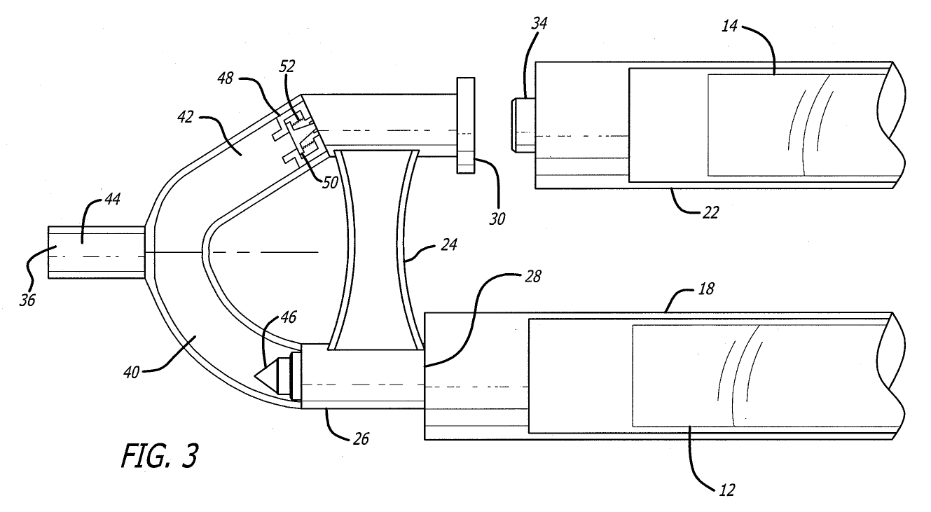

FIG. 3 shows an alternate embodiment of the assembly 10′. The second one-way valve 48′ may only allow fluid flow into the second syringe 14. By way of example, the second one-way valve may be a disk valve that includes a disk 50 biased into a closed position by a spring 52 or some other biasing element. The spring 52 should have a biasing force that prevents fluid in the first syringe 12 from flowing into the second syringe 14 when the first plunger 16 is depressed by a user.

The assembly 10′ can be used to both aspirate and inject fluid into a patient. By way of example, the needle may be inserted into a patient and the second plunger 20 may be pulled to aspirate patient fluid. The second one-way valve 48′ allows fluid to flow into the second syringe 14. The first plunger 16 may then be depressed to inject medical fluid, such as a steroid, into the patient. The medical fluid flows through the one-way valve 46. The second one-way valve 48′ prevents flow of the medical fluid into the second syringe 14.

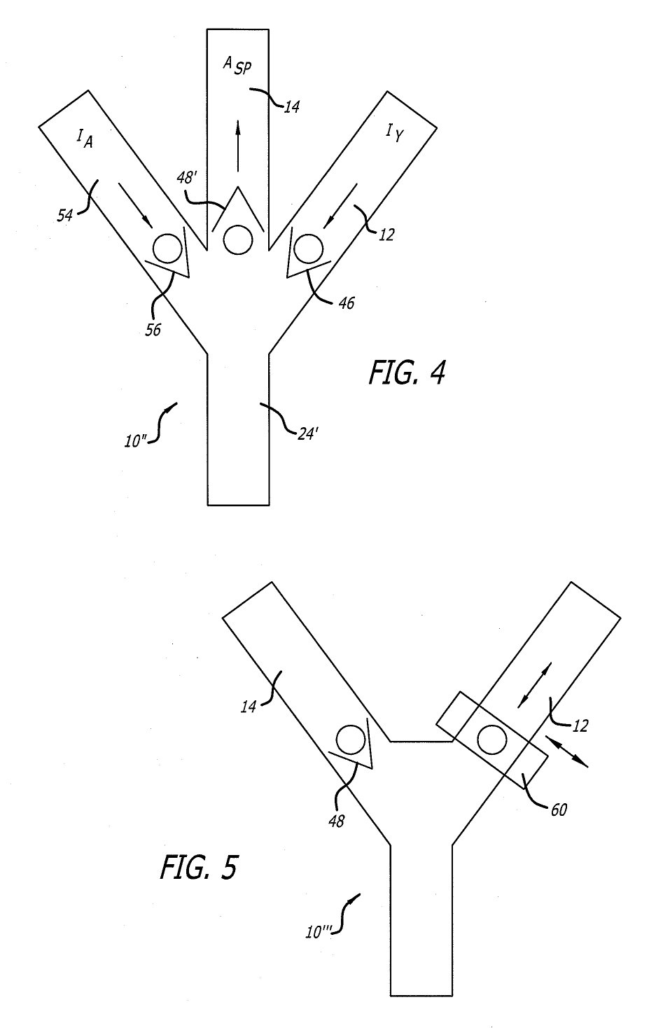

FIG. 4 shows another embodiment of an assembly 10″ that has a third syringe 54 and a third one-way valve 56 located in a connector valve assembly 24′. The third one-way valve 56 allows fluid to flow from the third syringe 54 into the needle (not shown). The assembly 10″ allows a user to aspirate fluid and inject two different medical fluids into a patient. By way of example, the first syringe 12 can be used to inject an anesthetic into a patient. The second syringe 14 is then used to aspirate fluid from a patient. Finally, the third syringe 54 is used to inject a steroid into the patient.

FIG. 5 shows an illustration of a syringe assembly 10″′ with two syringes 12 and 14. The first syringe 12 includes a two-way valve 60 that can be actuated to either allow flow out of the syringe 12 when in a first position, or flow into the syringe 12 when in a second position. By way of example, the valve 60 may be a manually stopcock type valve assembly. The second syringe 14 may include a one-way valve 48 that only allows fluid to flow out of the syringe 14.

The syringe assembly 10″′ allows for the injection of two different fluids and the aspiration of fluid with one device. For example, an anesthetic can be ejected from the first syringe 12 when the valve 60 is in one position. The valve 60 can then be moved to another position to allow patient fluid to be aspirated into the first syringe 12. A second medical fluid such as a steroid can then be ejected from the second syringe 14.

Although embodiments with 2 and 3 valves and syringes are shown and described, it is to be understood that the assembly may have any number of valves/syringes. Additionally, although automated valves are shown and described, it is to be understood that the valves can be configured to be manually opened or closed by the user.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A connector valve assembly for a plurality of syringes, comprising:
a housing that includes a pair of proximal ports, and a distal port in fluid communication with said proximal ports through first and second proximal channels;
a first one-way valve that is located within said first proximal channel and allows fluid flow in a first direction; and,
a second one-way valve located within said second proximal channel that allows fluid flow in a second direction opposite from said first direction.

2. The assembly of claim 1, wherein said second one-way valve is a disk valve.

3. The assembly of claim 1, further comprising third proximal port, a third proximal channel in fluid communication with said distal port a third one-way valve that is located within said third proximal channel and allows fluid to flow in the first direction.

4. The assembly of claim 1, further comprising a needle that is attached to said housing and is in fluid communication with said distal port.

5. The assembly of claim 1, wherein said proximal ports include attachment features to attach said housing to the syringes.

6. A syringe assembly, comprising:
a first syringe;
a second syringe;
a connector housing that is attached to said first and second syringes, said connector housing includes a first proximal port coupled to said first syringe, a second proximal port coupled to said second syringe, and a distal port in fluid communication with said first and second proximal ports through first and second proximal channels;
a first one-way valve that is located within said first proximal channel and allows fluid to flow out of said first syringe; and,
a second one-way valve that is located within said second proximal channel and allows fluid to flow into said second syringe.

7. The assembly of claim 6, wherein said second one-way valve is a disk valve.

8. The assembly of claim 6, further comprising third proximal port, a third proximal channel in fluid communication with said distal port a third one-way valve that is located within said third proximal channel and allows fluid to flow in the first direction.

9. The assembly of claim 6, further comprising a needle that is attached to said connector housing and is in fluid communication with said distal port.

10. A method for aspirating a fluid and injecting a medical fluid into a patient, comprising:
attaching a connector valve assembly to a first syringe that contains a first medical fluid and a second syringe, the connector valve assembly including a first one-way valve and a second one-way valve, and a needle extends from the connector valve assembly;
inserting the needle into a patient;
pulling the second syringe to aspirate fluid from the patient, the fluid flowing through the second one-way valve and into the second syringe; and,
pressing the first syringe to inject the medical fluid into the patient, the medical fluid flowing through the first one-way valve.

11. The method of claim 10, wherein the medical fluid is a steroid.

12. A method for aspirating a fluid and injecting two medical fluids into a patient, comprising:
attaching a connector valve assembly to a first syringe that contains a first medical fluid, a second syringe, and a third syringe that contains a second medical fluid, the connector valve assembly including a first one-way valve, a second one-way valve, a third one-way valve, and a needle extends from the connector valve assembly;
inserting the needle into a patient;
pressing the first syringe to inject the first medical fluid into the patient, the first medical fluid flowing through the first one-way valve;
pulling the second syringe to aspirate fluid from the patient, the fluid flowing through the second one-way valve and into the second syringe; and,
pressing the third syringe to inject the second medical fluid into the patient, the second medical fluid flowing through the third one-way valve.

13. The method of claim 12, wherein the first medical fluid is an anesthetic and the second medical fluid is a steroid.

14. A connector valve assembly for a plurality of syringes, comprising:
a housing that includes a pair of proximal ports, and a distal port in fluid communication with said proximal ports through first and second proximal channels;
a two-way valve that is located within said first proximal channel; and,
a one-way valve located within said second proximal channel.

15. The assembly of claim 14, wherein said two-way valve provides fluid communication through said first proximal channel in a first direction when in a first position, and allows fluid communication through said first proximal in a second opposite direction when in a second position.

16. The assembly of claim 14, further comprising a needle that is attached to said housing and is in fluid communication with said distal port.

17. The assembly of claim 14, wherein said proximal ports include attachment features to attach said housing to the syringes.

18. A syringe assembly, comprising:
a first syringe;
a second syringe;
a connector housing that is attached to said first and second syringes, said connector housing includes a first proximal port coupled to said first syringe, a second proximal port coupled to said second syringe, and a distal port in fluid communication with said first and second proximal ports through first and second proximal channels;
a two-way valve that is located within said first proximal channel; and,
a one-way valve that is located within said second proximal channel.

19. The assembly of claim 17, wherein said two-way valve provides fluid communication through said first proximal channel in a first direction when in a first position, and allows fluid communication through said first proximal in a second opposite direction when in a second position.

20. A method for aspirating a fluid and injecting a medical fluid into a patient, comprising:
attaching a connector valve assembly to a first syringe that contains a first medical fluid and a second syringe that contains a second medical fluid, the connector valve assembly including a two-way valve and a one-way valve, and a needle extends from the connector valve assembly, the two-way valve is in a first position;
inserting the needle into a patient;
pushing the first syringe to inject the first medical fluid into the patient;
switching the two-way valve to a second position;
pulling the first syringe to aspirate fluid from the patient into the first syringe; and,
pressing the second syringe to inject the second medical fluid into the patient.
